(19) United States
(12) Patent Application Publication
(10) Pub. No.: US 2004/0243135 A1
(43) Pub. Date: Dec. 2, 2004

(54) Hand drill

(76) Inventor: Tomoaki Koseki, Chiyoda-ku (JP)

Correspondence Address: Tomoaki Koseki, 17-2, Sotokanda 2-chome, Chiyoda-ku,Tokyo, 101-0021, JP

(21) Appl. No.: 10/776,741

(22) Filed: Feb. 9, 2004

(30) Foreign Application Priority Data
May 28, 2003 (JP) JP 2003-151609

(51) Int. Cl. A61B 17/00

(52) U.S. Cl. 606/80

(57) ABSTRACT

A hand drill with the shape of the drill blade tip having blades on the two edges of a shaved-powder ditch which is applied in a slanted straight line, and the structure of the drill is made so that the two edges of the shaved powder ditch is arranged gradually into acute angle towards the rotating direction. Also, the tip end of a round pillar material is press processed and blades are attached, forming a two-blade shape. A hook hole is applied at the side of the tip for pulling up a suture wire. The grip part and the drill axis part are connected with insert mould processing method, and the grip part is in a shape with uneven surface for smooth fitting in the palm. With the foresaid features, the resistance from the hole side due to hand trembling and the resistance at the time of pulling the drill out of the hole are reduced, enabling sufficient intensity and piercing force achieved in a short time, providing a hand drill that discharges shaved powder from bone tissue smoothly. This invention offers a hand drill for sternum suture, having a sharp cutting feature with a small cross section, avoiding inconveniences of breakage, acquiring space to discharge shaved powdered bone, and the drill can be pulled out easily.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC or REFERENCE TO A “MICROFICHE APPENDIX”

Not Applicable

### BACKGROUND OF THE INVENTION

(1) Field of the Invention

This invention relates to a hand drill to bore a hole manually.

(2) Description of Related Art including information disclosed under 37 CFR 1.97 and 1.98

A drill is usually in a shape of spiral with ditches made in the foreside in bidirectional ways from the tip, and the blade is attached only to the tip end. A half-moon drill has one edge of the cone-shape shaved off from the center of the tip and made as the blade. A gimlet has a simple structure with its center part of the square pillar material cut out into a foursided cone.

There are proposals for some blade shapes for the hand drill to bore large holes (patent documents 1 and 2). There are proposals for the positioning and fixing of the drilling (patent documents 3, 4 and 5).

Cardiovascular surgeries are usually conducted after median incision in vertical direction of the sternum located at the center of the chest, and the sternum is bound with stainless wire and such material after the operation. One method to pierce a piece of wire through the sternum is to hold with a needle holder a needle connected to the tip end of a wire and pierce the bone, or utilize a sternum piercing device and pass and pull up the wire at the hole at the end. (patent documents 6 and 7)

Patent document 1

Patent publication number Heisei 9-29524

Patent document 2

Patent publication number Heisei 9-29526

Patent document 3

Patent publication number Heisei 6-270005

Patent document 4

Patent publication number Heisei 7-80712

Patent document 5

Patent publication 2003-39217 (p2003-39217A)

Patent document 6

Patent Application 2000-045190

Patent document 7

Patent Application 2000-309910

A conventional drill is made with a blade attached at the tip, and using such type of drill makes the surgeon's hand tremble when drilling a hole manually, and there occurs strong resistance at the sides of the hole. Also when the surgeon tries to pull out the drill after drilling, the spiral part is caught in the tissue, and the surgeon often has to pull with strength or pry the drill out of the hole.

In the case of a half-moon drill, the drill takes almost half the volume of the hole diameter, which lowers the intensity of the drill, so such a half-moon drill is not suitable when the tissue is hard and stiff, or when the drilling needs to be done deeply. When the volume of the shave-off part is decreased, the shaved powder substances are accumulated within the hole, and there is more burden on the drill. Depending on the direction of the drill rotation, the shaving is done with just one blade, and thus the piercing strength weakens.

A gimlet simply has the center portion of the square pillar material cut out in four-sided cone, and therefore the cutting strength is extremely weak.

The structure of a bone is such that hard cortical bone covers a relatively flexible cancellous bone that includes much blood. When the bone is pierced with a device with a wide cutting section, the bone tissue starts to bleed and it takes time and labor for hemostasis. A sharp cutting feature with small cutting section is required, but a simple cutting face on the tip end as in a conventional drilling apparatus would not pierce through the cortical bone easily in a short time, and sometimes such drilling apparatus breaks, causing inconveniences.

When drilling a hole in a bone tissue, no space is provided to discharge the shaved powdered bone when using a conventional piercing apparatus, and due to blockage, the piercing process had to overcome further resistance.

When using a sternum piercing apparatus, it is pierced into the sternum, and the suture wire is passed and then pulled out at the hole at the tip end, but there is a strong resistance when pulling out the wire, and the wire cannot be pulled out easily by a surgeon wearing blood smeared gloves.

### BRIEF SUMMARY OF THE INVENTION

To solve the above issues, this invention proposes the following. The shape of the blade at the end of the hand drill is pointed in a taper form, providing a shaved-powder ditch for to discharge the shaved powder substances in a slanted straight line towards the handle part from the tip end, the width of which is from the center of the tip to the other edge. Both sides of the shaved-powder ditch are made into blades. To minimize the friction resistance, both edges of the shaved-powder ditch in a taper form become gradually sharper towards the rotating direction, being away from the bone surface, to avoid contact with the bone surface.

The tip end of the circular pillar form drill becomes bi-plane taper form from the tip end to the fore side, and the periphery has blades attached. The peak line of the tip end is slanted from both edges to the center, and blades are attached.

The center of the peak line is made into a ditch in a perpendicular direction, forming two blades. A straight linear slit is applied from the bottom end of the bi-plane taper form to the longitudinal direction.

The shape of the tip blade is a two-blade style in a fork shape, and the tip ends of the two-blade are attached to slant from the outside to inwards, and the base part of the fork shape two-blades is made so that one of the blades is slanted from the foreside towards the tip end.

This invention proposes a sternum suture drill in which a hook ditch is applied at the side of the tip end, to pull up the suture wire.

This invention proposes a hand drill with the pole shape grip part connected to the drill axis perpendicularly, and the grip part has uneven surface to enable the gripping to be done without forming any gap between the palm and the grip, when the drill is held by a surgeon with a forefinger and a middle finger around the grip part making a fist.

A T-shape plate form grip is attached to the tip end of the drill axis, and the surface of the T-shape plate has dimples and hollow parts as a slip proof measure.

A metal pole material is used to form the tip end with press processing, then the blades are attached with a grinder, and the tip part is inserted with pressure into the core metal applied as rotation-proof and reinforcement, and then the grip part and the core metal part are connected with resin insert moulding processing method.

### BRIEF DESCRIPTION OF THE SEVERAL VIEWS OF THE DRAWINGS

FIG. 1 is an external drawing of a sternum suture drill.

FIG. 2 is an enlarged drawing of the sternum suture drill tip.

FIG. 3 is a drawing of a sternum suture drill during usage.

FIG. 4 is an enlarged drawing of the sternum suture drill blade tip.

FIG. 5 is a drawing of a drill with a T-shaped plate form grip.

FIG. 6 is an enlarged drawing of a fork form two-blades.

### DETAILED DESCRIPTION OF THE INVENTION

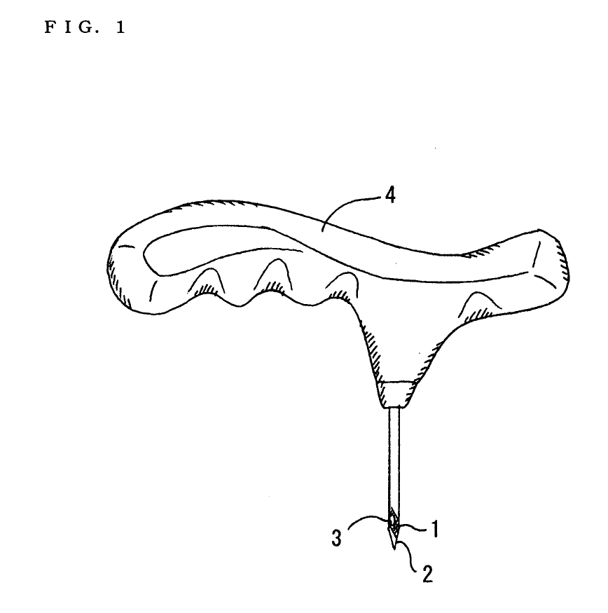

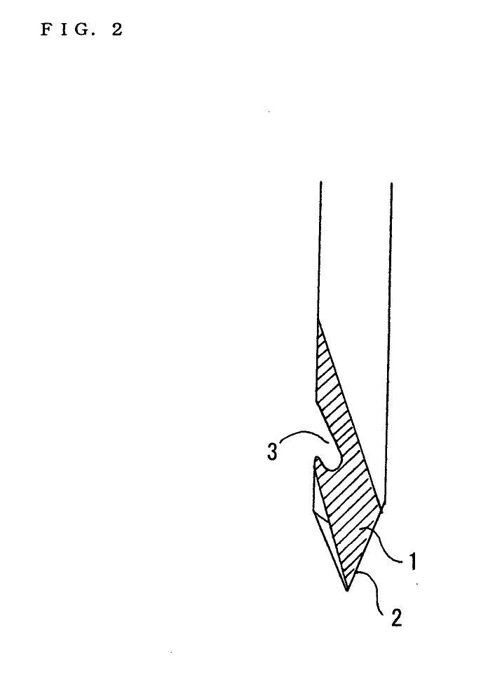

Hereafter, a description of the preferred embodiments of this invention is made in reference to drawings. FIG. 1 and FIG. 2 (enlarged drawing of the tip part) show one embodiment of this hand drill invention, showing a sternum suture drill. Usually, a high speed steel or super carbide steel is used for the material of a drill, but in the case of medical drills, stainless SUS 304, 330 or 420 and such are selected from the viewpoint of rustproof feature and break proof aspect. The edge of the blade as shown in the drawing is sharpened in a taper form, and a ditch for shaved powder is applied with press processing method in a diagonal straight line from the tip end to fore side. The two edges of the shave-powder ditch 1 are made into blades. The shaved-powder ditch 1 lessens the burden on the drill by effectively discharging the shaved powder, shaved at the drill tip end. The shaved-powder ditch 1 can be applied to the root part, made in parallel to the material, but if the whole of the drill is made in a plate form, the break proof intensity would decrease. To minimize the friction resistance, the two edges of the shaved-powder ditch at the tip taper part have blades attached. The blade 2 gradually becomes sharper towards the end of the blade in the rotating direction, made in a structure to be away from the bone surface to avoid contact with the bone surface. Hook hole 3 is applied to the side of the tip end with press processing method, to pull up the suture wire. Hook hole 3 can be made without any concern of damage when made by avoiding the center line of the drill axis. The drill tip end is inserted with pressure into core metal which is provided for anti-rotation and for reinforcement of the drill, and grip 4 is connected with the core metal with insert moulding method. It is preferable to utilize ABS, polyethylene, polystyrene, PET and such resin for the material of grip 4, having tolerance to radiation sterilization. When wrinkle processing is applied to the surface, such process will provide slip proof feature.

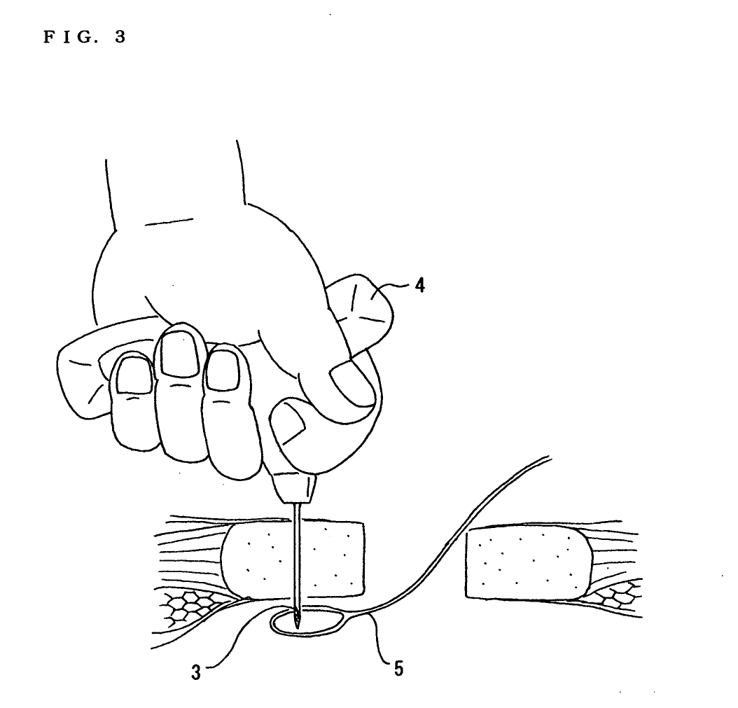

FIG. 3 is a drawing of a hand drill during usage, When the drill axis part is gripped between the forefinger and the middle finger and the hand is made into a fist, grip 4 fits smoothly into the palm, enabling smooth transmission of physical strength when inserting the drill into the sternum and when pulling the drill out of the sternum. After the drill is pierced into the sternum, hook hole 3 is turned to the opposite direction as the sternum incision side, the ring part of ring-attached wire 5 is hooked into hook hole 3, the drill is pulled up at the same time and the wire can pierce through the sternum speedily and easily.

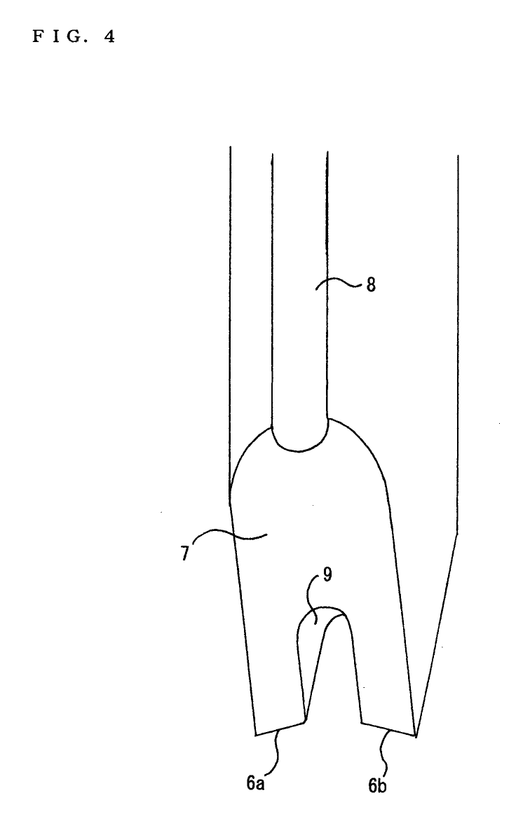

FIG. 4 is an enlarged drawing of the drill blade tip according to claims 2, 4, 5, 7 and 10. The tip of the round pillar material is crushed into a bi-plane taper form 2 with the first press processing method, and the end is formed like a driver tip end. A projection for slit 3 can be made onto the press mould, to form slit 3 in a longitudinal direction from the bottom end of bi-plane taper 2 at the time of press processing. Slit 3 can be used to discharge shaved powder, but can also lower the resistance caused while utilizing the drill, due to negative pressure when the drill is moved up and down, as blood, body fluid and other such substances permeate the hole when holes are bored into bones during surgery. Next, with second press processing method, ditch 4 is stamped out to the vertical direction of the peak line that is slanted, and peak line center. The drill tip end becomes two-blades 1a and 1b divided into two at the end. Lastly, blades are attached to the bi-plane taper peripheral part and to the peak line of the tip end. The two blades 1a and 1b have sharp edges turning either way, so this will have double the sharpness compared to one blade. When used in a surgery, this sharpness would lead to shortening of surgery time. This can be turned in the same direction in 360 degrees, but when this is turned 180 degrees in one direction and 180 degrees in the other direction while pressing, the piercing can be done at an even shorter time. As blades are attached to the bi-plane taper peripheral part 2, it becomes possible to shave off the hole sides, minimizing the resistance from hand trembling, and the drill can be pulled out easily after drilling.

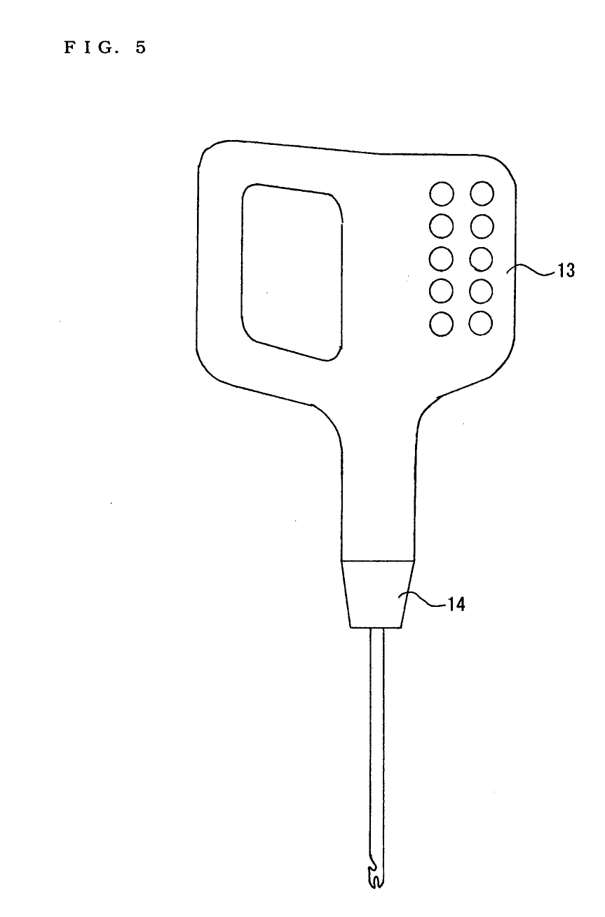

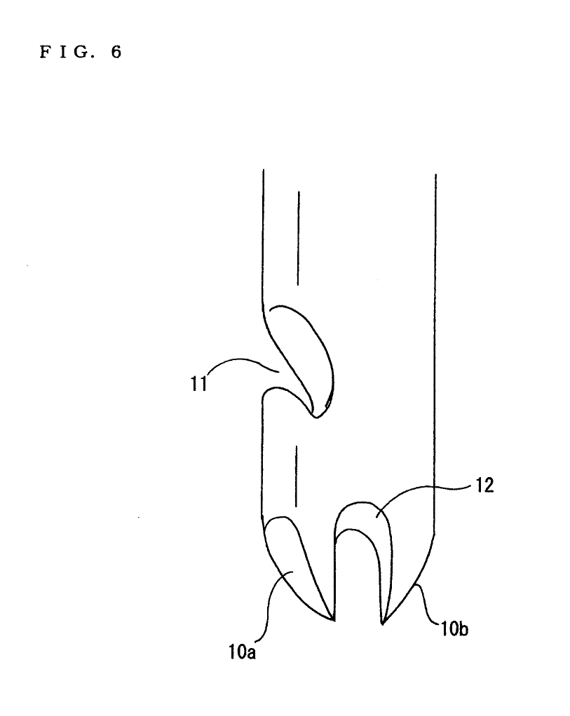

FIGS. 5 and 6 (enlarged drawing of the tip end) show embodiments of this invention, of a sternum suture drill according to claims 3, 4, 8 and 11. In a case of a cardiovascular surgery, this can be used as a drill to pierce a hole in which a wire for sternum suture is to be passed through, wherein the form of the tip blades are in a fork style two-blades 10a and 10b. The tip ends of the two-blades 10a and 10b are attached so that the blades will slant from the outside inwards. As blades are attached to the tip end and to the peripheral, the sharpness is highly improved compared to a simple gimlet type drill. One side of the base part of the fork form of the two-blades 10a and 10b is made into a slope 12 from the foreside to the tip. A hook ditch 11 is applied at the side closer to the tip, to pull up the suture wire. With this feature, it is possible to pull up the wire with a ring in just one movement, leading to shortening of surgery time. The material for the drill part is SUS304 which has a stronger hardness due to the press processing, and this is suitable as a material without any concern of breakage. Grip 13 is in a T-shape plate form, which is inserted and moulded into the drill axis part. The grip is made to fit the palm smoothly enabling easier grip to put strength in the vertical direction. The surface of the grip has linear dimples processed on half the surface and hollows processed on the other half for slip proof. ABS, PP, PE and such resin are suitable for the material for the grip part. The manufacturing method for this invention is, first, forming the tip from a pole material with press processing, then blades are attached with a grinder, then that part is inserted with pressure into the core metal 14 for rotation-proof and reinforcement, then grip 13 and core metal 14 are connected and formed with resin insert mould processing method.

As explained above, compared to conventional sternum piercing apparatuses, this invention enables much improvement of the sharpness, and eliminates the inconvenience of breakage. By providing the shaved-powder ditch, the shaved powder at the tip can be discharged efficiently, and also decreases the burden on the drill as a whole. This invention enables the surgeon to shave the sides of the hole, minimizing the resistance due to hand trembling, and it is easier to pull out the drill after drilling.

With the combination of press processing and insert mould processing methods, manual manufacturing processes can be omitted, enabling a large amount of cost cut. Furthermore, as majority of the drill is made of resin, the volume and weight of the total infectious medical waste material will decrease, contributing to the reduction of waste processing expense.

The grip is made to fit the palm, and it is easier to put more strength onto the drill. When used together with a wire with a ring, this invention enables speedy and easier piercing of wire through the sternum, thus shortening the surgery time.

1. A hand drill for manual usage to pierce holes, wherein the shape of the tip blade is sharpened in a tapered form, a shaved-powder ditch is applied in a slanted way from the tip end towards the foreside with the width of the center of the tip to one edge, with the structure of both edges of the shaved-powder ditch are blades, and the two edges of the shaved-powder ditch in the tapered form becomes gradually into acute angle in the rotating direction, so that the hand drill will move away from the surface of the bone avoiding contact with the bone surface.

2. A manual hole piercing hand drill, wherein the tip end of the round pillar form drill is made in a bi-plane taper form from the tip to the foreside, blades are attached on the peripheral of the drill, the peak line of the tip is slanted from the two edges towards the center onto which a blade is attached, the center part of the peak line has a ditch in the vertical direction forming two-blades, and a straight linear slit is applied in the longitudinal direction from bottom part of the bi-plane taper form.

3. A manual hole piercing hand drill, wherein the form of the tip blade is a two-blade form in a fork shape, with the feature of the two-blades being arranged so that the tip of the two-blades slant from the outside inwards, and the base part of the fork shape of the two-blades has one side made in a slope from the foreside to the tip.

4. A hand drill for sternum suture surgery of the hand drill according to claims 1, 2 and 3, to pass sternum suture wire through the sternum, wherein a hook ditch is formed at the side of the tip for pulling up the suture wire.

5. A hand drill for sternum suture surgery of the hand drill according to claims 1, 2 and 3, wherein the pole shape grip part is connected to the drill axis part perpendicularly, with the feature of the grip part in a smooth fitting form for the palm without any gap when held inside a fist with the drill axis part being gripped between the forefinger and a middle finger, enabling more strength to be transmitted when a hole is pierced with this drill manually.

6. A hand drill for sternum suture according to claim 4, wherein the pole shape grip part is connected to the drill axis part perpendicularly, with the feature of the grip part in a smooth fitting form for the palm without any gap when held inside a fist with the drill axis part being gripped between the forefinger and a middle finger, enabling more strength to be transmitted when a hole is pierced into a sternum with this drill manually.

7. A hand drill according to claims 1, 2 and 3, wherein the T-shape plate form grip is connected to the drill axis part, and the surface of the T-shape plate having dimples and hollows applied for slip proof features.

8. A hand drill for sternum suture according to claim 4, wherein the T-shape plate grip is connected to the drill axis part, and the surface of the T-shape plate having dimples and hollows applied for slip proof features.

9. A manufacturing method for a hand drill according to claim 5, when manufacturing a hand drill depicted above, wherein a metal pole material is utilized to form the tip with press processing method, blades are attached with a grinder, then the tip is inserted with pressure into a core metal for prevention of rotation and for reinforcement, and then the grip part and the core metal part are connected and formed with resin insert mould processing method.

10. A manufacturing method for a hand drill according to claim 7, for manufacturing a hand drill depicted above, wherein a metal pole material is utilized to form the tip with press processing method, blades are attached with a grinder, then the tip is inserted with pressure into the core metal for prevention of rotation and for reinforcement, and then the grip part and the core metal part are connected and formed with resin insert mould processing method.

11. A manufacturing method for a hand drill according to claims 6 and 8, when manufacturing a hand drill depicted above, wherein a metal pole material is utilized to form the tip with press processing method, blades are attached with a grinder, then the tip is inserted with pressure into the core metal for prevention of rotation and for reinforcement, and then the grip part and the core metal part are connected and formed with resin insert mould processing method.
